(19) United States
(12) Patent Application Publication
(10) Pub. No.: US 2011/0196299 A1
(43) Pub. Date: Aug. 11, 2011

(54) STRUCTURE OF NEEDLE FOR SYRINGE

(76) Inventor: Youn-Sung Kim, Daejeon (KR)

(21) Appl. No.: 13/123,853

(22) PCT Filed: Oct. 25, 2008
(86) PCT No.: PCT/KR2008/005966

§ 371 Date: Apr. 12, 2011

(51) Int. Cl. A61M 5/42

(52) U.S. Cl. 604/112

(57) ABSTRACT

There is provided a structure of a needle for a syringe and, more particularly, to a needle connected to a medical syringe variously used for an intramuscular injection, an intravenous injection, an injection of Ringer's solution, or the others, which comprises a groove or protrusion in diverse shapes formed on the surface of the needle, whereby the needle is easily inserted to reduce pain and irritation when an injection is administered and the needle is prevented from being off an injection direction to inject medicine into a desired part, without any error when injections are administered into a muscle and a vein.

### TECHNICAL FIELD

The present invention relates to a structure of a needle for a syringe and, more particularly, to a structure of a needle connected to a medical syringe variously used for an intramuscular injection, an intravenous injection, an injection of Ringer's solution, or the others, which comprises a groove or protrusion in diverse shapes formed on the surface of the needle, whereby the needle is easily inserted to reduce pain and irritation when an injection is administered and the needle is prevented from being off an injection direction to inject medicine into a desired part, without any error, when injections are administered into a muscle and a vein.

### BACKGROUND ART

Generally, to prevent those handling syringes for medical treatment from being pricked by an infected syringe by accident, safe syringes are used in most medical agencies.

As illustrated in FIG. 1, a general syringe comprises: a cylinder 1 with a connection tube 2 being different in thickness formed at the front of the cylinder 1 and a handle flange 8 protruding toward both sides to receive fingers of a user formed at the rear of the cylinder 1; a piston 3 inserted into the cylinder 1 so as to be movable forward or backward, along a length direction of the cylinder 1; a plunger 4 connected to the front of the piston 3 and moved in airtight contact with the cylinder 1 as the piston 3 is moved forward or backward, to provide a compressing force or a sucking force; a syringe needle fixing tube 5 connected to be attachable to or detachable from the connection tube 2; a syringe needle 6 fixed to the syringe needle fixing tube 5; and a syringe needle safety cap 7 covering the syringe needle 6 and connected to be attachable to or detachable from the syringe needle fixing tube 5.

The syringe needles have various shapes. Issued U.S. Pat. No. 2,409,979 (R. L. Huber) discloses a syringe needle for treatment, which will be described with reference to FIGS. 2 and 3.

As illustrated in FIGS. 2 and 3, in the syringe needle, an injection solution path 11 is formed lengthwise at the center of a body 6 which is nearly round-shaped in section. A curved part 15 is formed so as to be bent at a predetermined position of the body 6 and to be progressively smaller in cross section toward the front. A cutting edge 17 is formed around an injecting opening of the injection solution path 11. A beveled part 18 is formed to be extended at a predetermined angle from the cutting edge 17 to the curved part 15.

The beveled part 18 makes the cutting edge 17 to be shaper so as to better slit a tissue when the needle penetrates the tissue. Further, a tip 16 is formed in a pointed shape at the part where the beveled part 18 and the curved part 15 contact with each other, enabling to easily penetrate the tissue.

### DISCLOSURE OF INVENTION

### Technical Problem

While the aforementioned conventional needle for medical treatment as described above is inserted into the body of a patient and advanced to a target part, the sharp cutting edge and the pointed tip may prick a blood vessel. As a result, serious bleeding may be caused or nerves may be injured so that serious aftereffects may follow or the patient may be in danger of his/her life. Moreover, sine the surface of the needle is made of metal material, the friction between the surface of the needle and the skin of a patient is not smooth, to cause pain so that patients have adverse to the treatment.

Therefore, the present invention has been made to solve the above problems, and it is an aspect of the present invention to provide a structure of a needle connected to a medical syringe variously used for an intramuscular injection, an intravenous injection, an injection of Ringer's solution, or the others, which comprises a groove or protrusion in diverse shapes formed on the surface of the needle, whereby the needle is easily inserted to reduce pain and irritation when an injection is administered and the needle is prevented from being off an injection direction to inject medicine into a desired part, without any error when injections are administered into a muscle and a vein.

### Technical Solution

In accordance with the present invention, the above and other aspects can be accomplished by a structure of a needle for a medical syringe which is used in the various fields, such as intramuscular injections, intravenous injections, injections of Ringer's solution or the others and which comprises:

a cylinder in which a multi-stage connection tube is formed at one end of the cylinder and a handle flange protruding toward both sides to receive fingers of a user is formed at the other end of the cylinder;

a piston inserted into the cylinder so as to move forward or backward, along a length direction of the cylinder, in which a plunger is formed at one end of the piston and is moved in airtight contact with the cylinder, together with the piston moved forward or backward inside the cylinder, to provide a compressing force and a sucking force;

a syringe needle fixing tube connected to be attachable to or detachable from the connection tube;

a syringe needle fixed to one end of the syringe needle fixing tube and inserted into a part of the body of a patient; and

a syringe needle safety cap covering the syringe needle and connected to be attachable to or detachable from the outer circumferential surface of the syringe needle fixing tube,

the structure of the syringe needle comprising: a groove or protrusion formed on the outer circumferential surface of the syringe needle, to relieve pain when an injection is applied to the patient and to make it easy to insert the needle into a part of the body to cause no irritation.

### Advantageous Effects

As described above, in accordance with the present invention, since the structure of a needle for a medical syringe variously used for an intramuscular injection, an intravenous injection, an injection of Ringer's solution, or the others, comprises the groove or protrusion in diverse shapes formed on the surface of the needle, there are the effects in that the needle is easily inserted to reduce pain and irritation when an injection is administered, and the needle is prevented from being off an injection direction to inject medicine into a desired part, without any error when injections are administered into a muscle and a vein.

### BRIEF DESCRIPTION OF THE DRAWINGS

These and other aspects and advantages of the present invention will become apparent and more readily appreciated from the following description of the embodiments, taken in conjunction with the accompanying drawings, in which:

FIG. 1 is an exploded perspective view of a conventional syringe;

FIG. 2 is a side view of the syringe of FIG. 1;

FIG. 3 is a plan view of the syringe of FIG. 1;

FIG. 4 is a perspective view of a syringe according to a first embodiment of the present invention;

FIG. 5 is a plan view of a needle for the syringe according to the first embodiment;

FIG. 6 is a plan view of a needle for a syringe according to a second embodiment of the present invention;

FIG. 7 is a plan view of a needle for a syringe according to a third embodiment of the present invention;

FIG. 8 shows sectional views of parts A-A in FIGS. 5 and 7;

FIG. 9 is a perspective view of a syringe according to a fourth embodiment of the present invention;

FIG. 10 is a plan view of a needle for the syringe according to the fourth embodiment;

FIG. 11 is a plan view of a needle for a syringe according to a fifth embodiment of the present invention;

FIG. 12 is a plan view of a needle for a syringe according to a sixth embodiment of the present invention; and

FIG. 13 shows a sectional view of parts A-A in FIGS. 10 and 12.

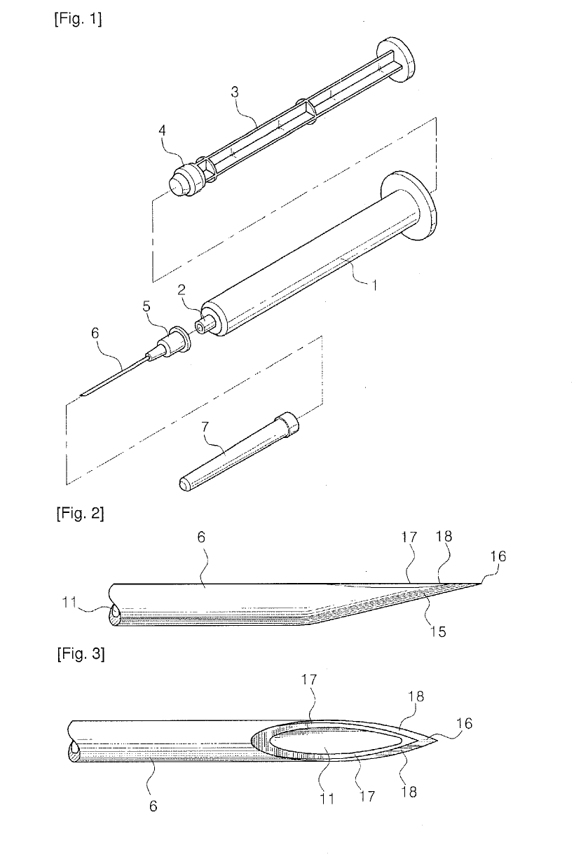

### BRIEF DESCRIPTION OF REFERENCE NUMBERS OF MAJOR ELEMENTS

10: syringe 20: cylinder
21: connection tube 22: handle flange
30: piston 31: plunger
40: syringe needle fixing tube 50: syringe needle
60: groove 70: syringe needle safety cap
80: protrusion

### BEST MODE FOR CARRYING OUT THE INVENTION

The present invention to achieve the above aspects has the characteristics as follows:

In accordance with an embodiment of the present invention, there is a structure of a needle for a medical syringe which is used in the various fields, such as intramuscular injections, intravenous injections, injections of Ringer's solution or the others and which comprises:

a cylinder in which a multi-stage connection tube is formed at one end of the cylinder and a handle flange protruding toward both sides to receive fingers of a user is formed at the other end of the cylinder;

a piston inserted into the cylinder so as to move forward or backward, along a length direction of the cylinder, in which a plunger is formed at one end of the piston and is moved in airtight contact with the cylinder, together with the piston moved forward or backward inside the cylinder, to provide a compressing force and a sucking force;

a syringe needle fixing tube connected to be attachable to or detachable from the connection tube;

a syringe needle fixed to one end of the syringe needle fixing tube and inserted into a part of the body of a patient; and

a syringe needle safety cap covering the syringe needle and connected to be attachable to or detachable from the outer circumferential surface of the syringe needle fixing tube,

and the structure of the syringe needle comprising: a groove formed on the outer circumferential surface of the syringe needle, thereby relieving pain when an injection is applied to the patient and making it easy to insert the needle into a part of the body, to cause no irritation.

In accordance with another embodiment of the present invention, there is a structure of a needle for a medical syringe which is used in the various fields, such as intramuscular injections, intravenous injections, injections of Ringer's solution or the others and which comprises:

a cylinder in which a multi-stage connection tube is formed at one end of the cylinder and a handle flange protruding toward both sides to receive fingers of a user is formed at the other end of the cylinder;

a piston inserted into the cylinder so as to move forward or backward, along a length direction of the cylinder, in which a plunger is formed at one end of the piston and is moved in airtight contact with the cylinder, together with the piston moved forward or backward inside the cylinder, to provide a compressing force and a sucking force;

a syringe needle fixing tube connected to be attachable to or detachable from the connection tube;

a syringe needle fixed to one end of the syringe needle fixing tube and inserted into a part of the body of a patient; and

a syringe needle safety cap covering the syringe needle and connected to be attachable to or detachable from the outer circumferential surface of the syringe needle fixing tube,

and the structure of the syringe needle comprising: a protrusion formed on the outer circumferential surface of the syringe needle, thereby relieving pain when an injection is applied to the patient and making it easy to insert the needle into a part of the body, to cause no irritation.

### MODE FOR THE INVENTION

The present invention will now be described more fully hereinafter with reference to the accompanying drawings, in which preferred embodiments of the invention are shown.

It will be understood that words or terms used in the specification and claims shall not be interpreted as the meaning defined in commonly used dictionaries. It will be further understood that the words or terms should be interpreted as having a meaning that is consistent with their meaning in the context of the relevant art and the technical idea of the invention, based on the principle that an inventor may properly define the meaning of the words or terms to best explain the invention.

As used herein, the singular forms “a”, “an” and “the” are intended to include the plural forms as well, unless the context clearly indicates otherwise.

The present invention may be embodied in different forms and should not be construed as limited to the embodiments set forth herein. Rather, these embodiments are provided as teaching examples of the invention. Therefore, it will be understood that the scope of the invention is intended to include various modifications and alternative arrangements within the capabilities of persons skilled in the art using presently known or future technologies and equivalents.

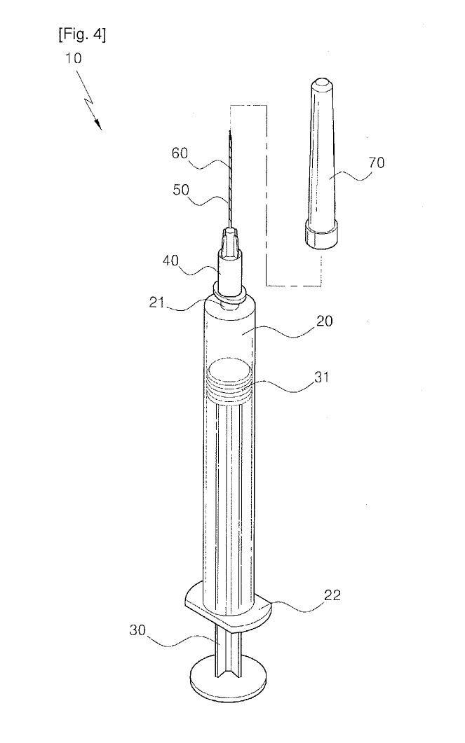

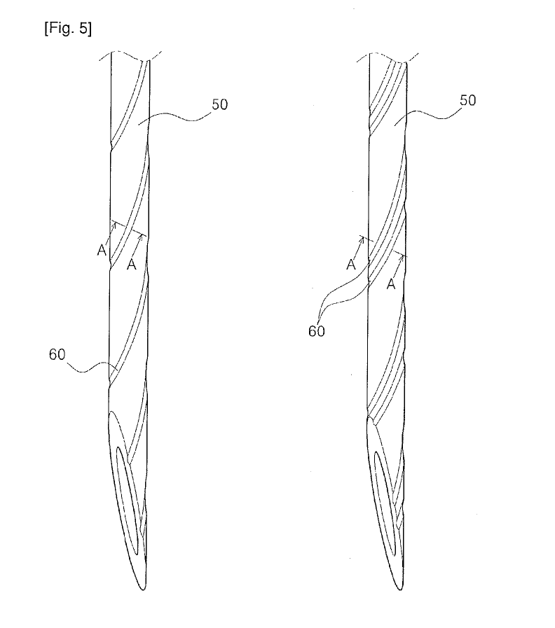

FIG. 4 is a perspective view of a syringe 10 according to a first embodiment of the present invention, and FIG. 5 is a plan view of a needle for the syringe according to the first embodiment.

As illustrated in FIG. 4, the syringe 10 comprises: a cylinder 20 in which a multi-stage connection tube 21 is formed at one end of the cylinder 20 and a handle flange 22 protruding toward both sides to receive fingers of a user is formed at the other end of the cylinder 20; a piston 30 inserted into the cylinder 20 so as to be moved forward or backward, along a length direction of the cylinder 20, in which a plunger 21 made of rubber material is positioned at one end of the piston 30 and is moved in airtight contact with the cylinder 20, together with the piston 30 moved forward or backward inside the cylinder 20, to provide a compressing force and a sucking force; a syringe needle fixing tube 40 connected to be attachable to or detachable from the connection tube 21; a syringe needle 50 fixed to one end of the syringe needle fixing tube 40 and inserted into a part of the body of a patient; and a syringe needle safety cap 70 covering the syringe needle 50 and connected to be attachable to or detachable from the outer circumferential surface of the syringe needle fixing tube 40.

The syringe 10 is the same as all syringes (disposable syringe, syringe for injection for R Ringer's solution, and others) which are variously used as a general medical instrument. Therefore, no further detailed description of the syringe 10 will be presented.

As illustrated in FIG. 5, a groove 60 is formed on the outer circumferential surface of the syringe needle 50 according to the present invention, to relieve pain when an injection is applied to the patient and make it easy to insert the needle into a part of the body, thereby causing no irritation. The groove 60 is formed in a spiral shape on the outer circumferential surface of the syringe needle 50. A number of the grooves 60 in the spiral shape are formed in one or more lines and are spaced apart from one another at regular intervals lengthwise.

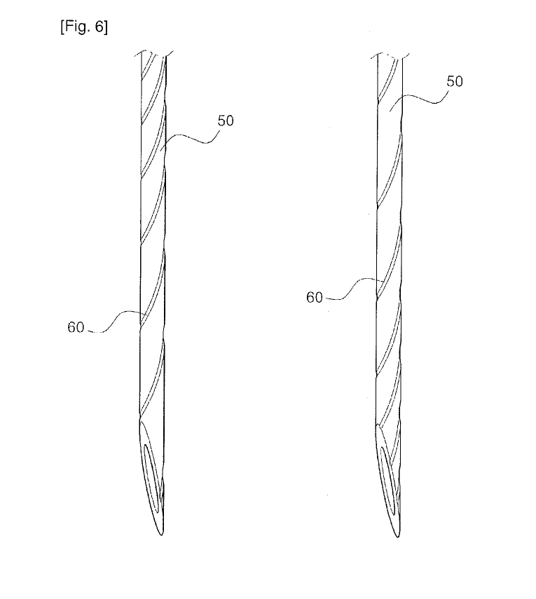

FIG. 6 is a plan view of a needle for a syringe according to a second embodiment of the present invention.

As illustrated in FIG. 6, a groove 60 is formed in a spiral shape (a screw shape) on the outer circumferential surface of a syringe needle 50. The groove 60 in the spiral shape is formed to be progressively narrower toward a pointed part of the syringe needle 50 in the interval of the groove 60. To the contrary, the groove 60 in the spiral shape may be formed to be progressively narrower toward an opposite part to the pointed part of the syringe needle 50 and progressively wider toward the pointed part of the syringe needle 50 in the interval of the groove 60.

FIG. 7 is a plan view of a needle for a syringe according to a third embodiment of the present invention.

As illustrated in FIG. 7, a number of grooves 60 are formed on the outer circumferential surface of a syringe needle 50 lengthwise. The interval between the grooves 60 is maintained to be consistent, thereby making it easy to insert the syringe needle 50 when an injection is applied to the body of a patient and reducing the pain felt by the patient. The grooves 60 are formed to be connected to the end of the syringe needle 50 in a straight line.

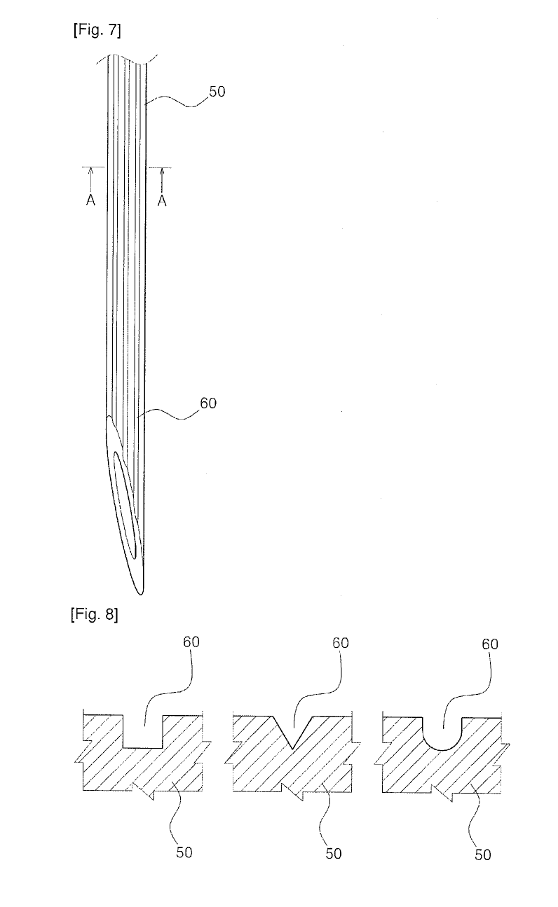

FIG. 8 shows sectional views of parts A-A in FIGS. 5 and 7.

As illustrated in FIG. 8, the section of the groove 60 in the parts A-A in FIGS. 5 and 7 has various shapes, such as a rectangular shape, an inverted triangular shape and a hemispheric shape.

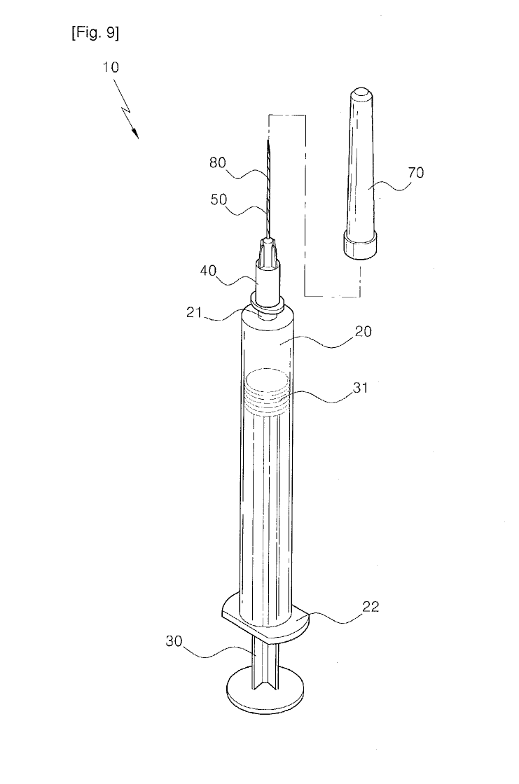

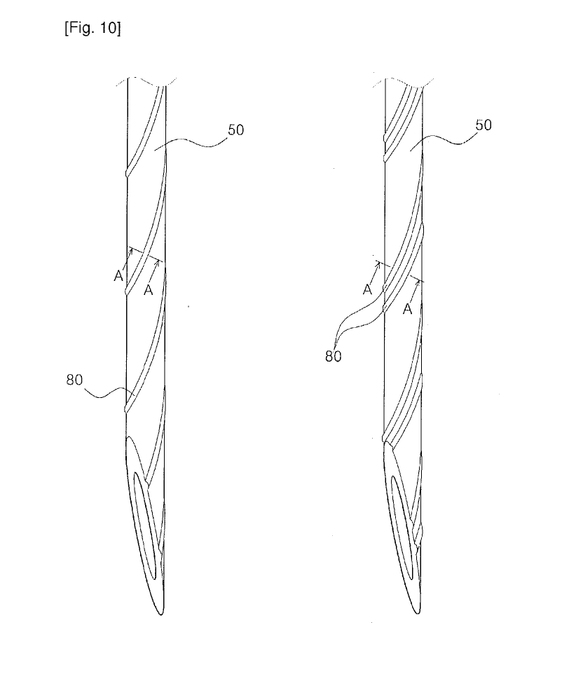

FIG. 9 is a perspective view of a syringe 10 according to a fourth embodiment of the present invention, and FIG. 10 is a plan view of a needle for the syringe 10 according to the fourth embodiment.

As illustrated in FIG. 9, the syringe 10 comprises: a cylinder 20 in which a multi-stage connection tube 21 is formed at one end of the cylinder 20 and a handle flange 22 protruding toward both sides to receive fingers of a user is formed at the other end of the cylinder 20; a piston 30 inserted into the cylinder 20 so as to be moved forward or backward, along a length direction of the cylinder 20, in which a plunger 21 made of rubber material is positioned at one end of the piston 30 and is moved in airtight contact with the cylinder 20, together with the piston 30 moved forward or backward inside the cylinder 20, to provide a compressing force and a sucking force; a syringe needle fixing tube 40 connected to be attachable to or detachable from the connection tube 21; a syringe needle 50 fixed to one end of the syringe needle fixing tube 40 and inserted into a part of the body of a patient; and a syringe needle safety cap 70 covering the syringe needle 50 and connected to be attachable to or detachable from the outer circumferential surface of the syringe needle fixing tube 40.

The syringe 10 is the same as all syringes (disposable syringe, syringe for injection for R Ringer's solution, and others) which are variously used as a general medical instrument. Therefore, no further detailed description of the syringe 10 will be presented.

As illustrated in FIG. 10, a protrusion 80 is formed on the outer circumferential surface of the syringe needle 50 according to the present invention, to relieve pain when an injection is applied to the patient and make it easy to insert the needle into a part of the body, thereby causing no irritation. The protrusion 80 is formed in a spiral shape on the outer circumferential surface of the syringe needle 50. A number of the protrusion 80 in the spiral shape are formed in one or more lines and are spaced apart from one another at regular intervals lengthwise.

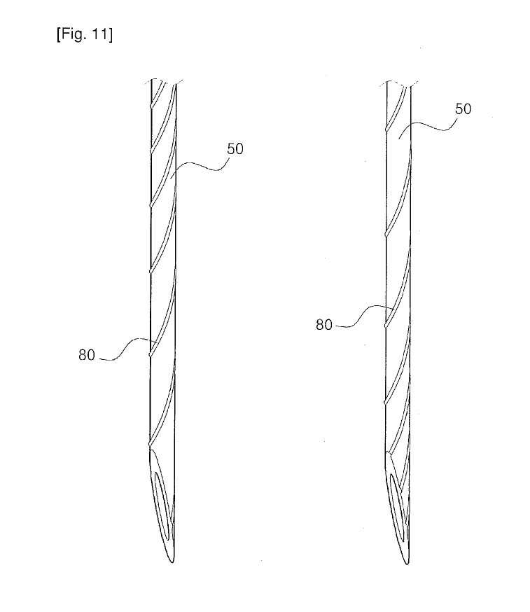

FIG. 11 is a plan view of a needle for a syringe according to a fifth embodiment of the present invention.

As illustrated in FIG. 11, a protrusion 80 is formed in a spiral shape (a screw shape) on the outer circumferential surface of a syringe needle 50. The protrusion 80 in the spiral shape is formed to be progressively narrower toward a pointed part of the syringe needle 50 in the interval of the protrusion 80. To the contrary, the protrusion 80 in the spiral shape may be formed to be progressively narrower toward an opposite part to the pointed part of the syringe needle 50 and progressively wider toward the pointed part of the syringe needle 50 in the interval of the protrusion 80.

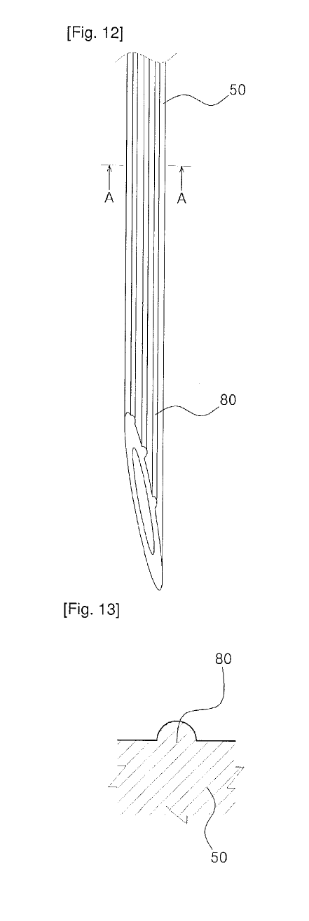

FIG. 12 is a plan view of a needle for a syringe according to a sixth embodiment of the present invention.

As illustrated in FIG. 12, a number of protrusions 80 are formed on the outer circumferential surface of a syringe needle 50 lengthwise. The interval between the protrusions 80 is maintained to be consistent, thereby making it easy to insert the syringe needle 50 when an injection is applied to the body of a patient and reducing the pain felt by the patient. The protrusions 80 are formed to be connected to the end of the syringe needle 50 in a straight line.

FIG. 13 shows a sectional view of parts A-A in FIGS. 10 and 12.

As illustrated in FIG. 13, the protrusion 80 in the parts A-A in FIGS. 10 and 12 protrudes in a hemispheric shape in section but it may be formed in various shapes.

1. A structure of a needle for a medical syringe, which is used in the various fields, such as intramuscular injections, intravenous injections, injections of Ringer's solution or the others and which comprises:
a cylinder in which a multi-stage connection tube is formed at one end of the cylinder and a handle flange protruding toward both sides to receive fingers of a user is formed at the other end of the cylinder;
a piston inserted into the cylinder so as to be moved forward or backward, along a length direction of the cylinder, in which a plunger is positioned at one end of the piston and is moved in airtight contact with the cylinder, together with the piston moved forward or backward inside the cylinder, to provide a compressing force and a sucking force;
a syringe needle fixing tube connected to be attachable to or detachable from the connection tube;
a syringe needle fixed to one end of the syringe needle fixing tube and inserted into a part of the body of a patient; and
a syringe needle safety cap covering the syringe needle and connected to be attachable to or detachable from the outer circumferential surface of the syringe needle fixing tube, the structure of a needle comprising:
a groove formed on the outer circumferential surface of the syringe needle, to relieve pain when an injection is applied to the patient and to make it easy to insert the syringe needle into a part of the body of the patient to cause no irritation.

2. A structure of a needle for a medical syringe, which is used in the various fields, such as intramuscular injections, intravenous injections, injections of Ringer's solution or the others, comprising:
a protrusion formed on the outer circumferential surface of a syringe needle, to relieve pain when an injection is applied to the patient and to make it easy to insert the syringe needle into a part of the body of the patient to cause no irritation.

3. The structure of a needle according to claim 2, wherein the medical syringe comprises:
a cylinder in which a multi-stage connection tube is formed at one end of the cylinder and a handle flange protruding toward both sides to receive fingers of a user is formed at the other end of the cylinder;
a piston inserted into the cylinder so as to be moved forward or backward, along a length direction of the cylinder, in which a plunger is positioned at one end of the piston and is moved in airtight contact with the cylinder, together with the piston moved forward or backward inside the cylinder, to provide a compressing force and a sucking force;
a syringe needle fixing tube connected to be attachable to or detachable from the connection tube;
a syringe needle fixed to one end of the syringe needle fixing tube and inserted into a part of the body of a patient; and
a syringe needle safety cap covering the syringe needle and connected to be attachable to or detachable from the outer circumferential surface of the syringe needle fixing tube.

4. The structure of a needle according to claim 1, wherein the groove or the protrusion is formed on the outer circumferential surface of the syringe needle in a spiral shape in one or more lines.

5. The structure of a needle according to claim 1, wherein a number of the grooves or a number of the protrusions are formed on the outer circumferential surface of the syringe needle lengthwise.

6. The structure of a needle according to claim 1, wherein the groove or the protrusion in the spiral shape are formed to be progressively narrower or wider toward an end of the syringe needle in the interval between the grooves or between the protrusions.

7. The structure of a needle according to claim 2, wherein the groove or the protrusion is formed on the outer circumferential surface of the syringe needle in a spiral shape in one or more lines.

8. The structure of a needle according to claim 2, wherein a number of the grooves or a number of the protrusions are formed on the outer circumferential surface of the syringe needle 1 lengthwise.

9. The structure of a needle according to claim 2, wherein the groove or the protrusion in the spiral shape are formed to be progressively narrower or wider toward an end of the syringe needle in the interval between the grooves or between the protrusions.
